(19) United States
(12) Patent Application Publication
(10) Pub. No.: US 2026/0029389 A1
(43) Pub. Date: Jan. 29, 2026

(54) ANALYTICAL PROCESS FOR DETECTING PEROXIDE-, HALOGEN OXOANION-, AND NITRATE-BASED EXPLOSIVES

(71) Applicant: Christian-Albrechts-Universität zu Kiel, Kiel (DE)

(72) Inventors: Rainer Herges, Kiel (DE); Morten Peters, Kiel (DE); Mike Brockmann, Kiel (DE)

(21) Appl. No.: 19/349,137

(22) Filed: Oct. 3, 2025

Related U.S. Application Data
(63) Continuation of application No. 17/291,389, now Pat. No. 12,436,135.

(30) Foreign Application Priority Data
Apr. 1, 2020 (EP) 20167599

(51) Int. Cl. G01N 31/22; G01N 21/78

(52) U.S. Cl. CPC G01N 31/228; G01N 21/78; G01N 31/227; Y10T 436/206664

(57) ABSTRACT

An analytical process, which preferably is a one-step process, for detecting in a sample peroxide-based explosives, and/or nitrate-based explosives, the process comprising contacting a sample suspected of containing a peroxide-based compound, especially a peroxide-based explosive, or a nitrate salt, especially a nitrate-based explosive, with a composition comprising a Ni-porphyrin, an acid and preferably an acid-stable solvent.

### PRIORITY CLAIM AND REFERENCE TO RELATED APPLICATION

The application is a continuation of and claims priority under 35 U.S.C. § 120 and all applicable statutes and treaties from prior pending U.S. Application Ser. Number U.S. Ser. No. 17/291,389, filed May 5, 2021, which application is a 35 U.S.C. 371 US National Phase and claims priority under 35 U.S.C. § 119, 35 U.S.C. 365(b) and all applicable statutes and treaties from prior PCT Application PCT/EP2021/058279, which was filed Mar. 30, 2021 which application claimed priority from European Patent Application EP 20167599.8, which was filed on Apr. 1, 2020. All US and PCT applications mentioned in this paragraph are incorporated by reference.

### FIELD

A field of the invention is explosives detection.

### BACKGROUND

Detection kits might contain several reagents or host compounds to cover a wider range of classes of explosives and to differentiate between them. Chemical methods usually need sample preparation. There are several methods for the chemical detection of nitrogen-based explosives; however, there is only a limited number of colorimetric detection methods for peroxide-based explosives:

WO 99/43846 (Ehud Keinan und Harel Itzhaky) describes a colorimetric method and kit for the detection of peroxide based explosives (cyclic peroxides), such as triacetone peroxide (TATP), diacetone peroxide (DADP) and hexamethylene triperoxide diamine (HMTD). Peroxide detection is based on a two-step process. The first step involves dissolution of the suspected material in an organic solvent and the hydrolysis of the cyclic peroxide with strong acids into acetone and hydrogen peroxide. In the second step, the acidic solution is neutralized with a buffer and the hydrogen peroxide is detected by the color change of a redox active dye oxidized by the hydrogen peroxide and a peroxidase enzyme. The patent includes a kit for the practical use of the invention including an organic solvent, a strong acid, a solution of the buffer, a peroxidase enzyme and the redox active dye. The reason for this rather complicated detection procedure is the fact that a strong acid is needed for the cleavage of the cyclic peroxide (explosive), however, the actual colorimetric detection can only be performed at neutral pH. Therefore, neutralization of the solution is necessary as an intermediate step.

Lin and Suslick, J. Am. Chem. Soc. 15519-15521 (2010) for colorimetrically detecting TATP describe a process in which a gaseous sample flows through a bed of the acid form of a sulfonated highly cross-linked polystyrene ion-exchange resin Amberlyst 15 to achieve acid hydrolysis, with subsequent contacting the reaction products with an array of redox dyes. Since the acid is immobilized in the porous, solid resin, and cleavage of the cyclic peroxide occurs in the gas phase inside the resin, a separate neutralization step is avoided. However, the disadvantage of this procedure is the fact, that only volatile explosives (TATP and DADP) can be detected. Moreover, to develop a sufficient vapour pressure for detection, TATP and DADP must be placed in a container or at least at a place with minimal air exchange. Hexamethylene triperoxide diamine (HMTD), which has a higher sublimation point and a lower vapor pressure as compared to TATP and DADP, cannot be detected. Advantage of the method is the fact that TATP can be discriminated from other oxidizing agents such as hydrogen peroxide and hydroperoxides by exposing the vapor after solid acid hydrolysis to an array of redox active dyes with different reactivities.

Amisar in WO 2005/089058 A2 described a method for the detection of chlorate, bromate and/or organic peroxides. The detection kit includes two containers, including an organic solvent, strong acid, an aromatic amine and a transition metal ion.

In a follow-up patent WO 2010/086834 Amisar describes a sequential procedure that includes several steps and several reagents to detect 1. perchlorates, 2. nitroaromatic compounds, 3. nitramines, nitroester, chlorate and bromate, 4. peroxides, 5. nitrates from the same sample.

Schulte-Ladbeck et al., Analyst 1152-1154 (2002) for detecting TATP or HMTD describe the addition of catalase to a liquid test sample for removal of free hydrogen peroxide, followed by extraction with acetonitrile to remove the catalyst, and subsequently irradiating the sample with UV in order to decompose the peroxide-based compounds, and in an enzyme-catalysed reaction colorimetrically detecting the hydrogen peroxide generated by the decomposition reaction.

U.S. Pat. No. 7,799,573 B2, Detection of explosives and other species, describes the detection of peroxide-based explosives by acid or light induced decomposition to hydrogen peroxide and subsequent detection of the hydrogen peroxide by reaction with oxalic acid esters and detection of the chemiluminescence thereof.

M. K. Chahal, M. Sankar, Dalton Trans. 2016, 45, 16404-16412 describes synthesis of Ni-porphyrins (Ni-tetra(4-hydroxyphenyl)porphyrins).

M. K. Chahal, M. Sankar, Dalton Trans.2017, 46, 11669-11678 describes the detection of cyanide (CN⁻), fluoride (F⁻) and picric acid using as sensor compound Ni-porphyrins (Ni-tetra(4-hydroxyphenyl)porphyrins).

Without relation to use in detecting explosives, Cong et al., Inorganic Chemistry 14361-14376 (2019) describes perhalogenated Ni-porphyrin compounds.

Without relation to use in detecting explosives, B. Du, A. Langlois, D. Fortin, C. Stern, P. D. Harvey, J. Clust. Sci. 2012, 23, 737-751 describes Ni-porphyrin compounds, which when substituted with an acid group have a pKₐ 4.2.

Without relation to use in detecting explosives, C. Matos, C. Ribeiro, L. R. Gomes, Med. Chem. Res. 2015, 24, 3885-3891 describes the interaction of an acid substituted porphyrin (meso-tetra(4-carboxyphenyl)porphine) with model membranes.

Optical detection methods of nitroaromatics, nitramines and nitrate esters are reviewed in “Optical explosives detection: from color changes to fluorescence turn-on, Germain, M. E.; Knapp, M. J. Chem. Soc. Rev. 2009, 38, 2543). Methods to detect nitrogen-based explosives such as inorganic nitrates, organic nitrates and nitramines have been described in: Method and kit for detecting explosives, Margalit, Y. Eur. Pat. Appl. (1994), EP 586125 A2 and U.S. Pat. No. 5,480,612.

WO 2008/130376 A2 (Determination of explosives including RDX) presents methods to detect nitramines, e.g. RDX and PETN using dyes such as 9,10-didehydroacridine or 9,10-dide-hydroanthracene derivatives that act as a hydride donors upon irradiation with UV light, reducing the nitramines, thereby being converted to fully conjugated acridine or anthracene derivatives. Detection is performed by observation of changes in absorption or emission.

WO 2013/022494 (Detection of analytes including nitro-containing analytes) describes a variation of WO 2008/130376 A2 wherein the decomposition products after irradiation with UV light react via electrophilic aromatic substitution of electron rich aromatic compounds. Detection is based on an optical signal, which may be a change in absorption or emission.

A. D. Jarczewski, P. Pruszynski, K. T. Leffek, Can. J. Chem. 1979, 57, 669-672. 2. S. A. H. Amas, H. J. Yallop, Analyst 196691, 336-337 describes that nitrotoluenes, e.g. 2,4-dinitrotoluene (DNT) or 2,4,6-trinitrotoluene (TNT), immediately give rise to a blue (DNT) or a purple color (TNT) upon treatment with an organic superbase.

None of these papers or patents describes a general colorimetric method (detection based on an optical signal) to detect peroxide-based explosives, oxyhalide anions and/or nitrate salts with the same reagent. None of the published methods reports on a colorimetric method to detect peroxide-based explosives (cyclic peroxides) in one stage without a separate activation step using acid, base or UV light.

### SUMMARY

A preferred embodiment provides an analytical process for detecting explosive compounds by a color reaction. A sample suspected of containing a peroxide-based compound, a nitrate-based compound, an oxoanion-based compound, an oxyhalide anion-based compound, a chlorate or a bromate is contacted with a composition comprising a Ni-porphyrin and a strong free acid having a pKₐ of −2.8 to 1.2. The existence of the color reaction is checked

### BRIEF DESCRIPTION OF THE DRAWINGS

The invention is described in greater detail by way of examples with reference to

FIG. 1 that shows UV spectra of the reaction product obtained by the analytical process. The UV spectra shown in FIG. 1 are superimposed of a composition according to the invention of Ni-tetrakis(trimethoxyphenyl)porphyrin and trifluoroacetic acid (TFA), of the reaction product of this composition according to the invention with TATP after 2.3 min as indicated, and of the reaction product of this composition according to the invention with TATP after 12.5 min. The UV spectra prove that the Soret and Q band, which are characteristic for the Ni-porphyrin, decrease in the presence of TATP and a broad band between 550 and 700 nm is built up, which band is typical for the porphyrin radical cation.

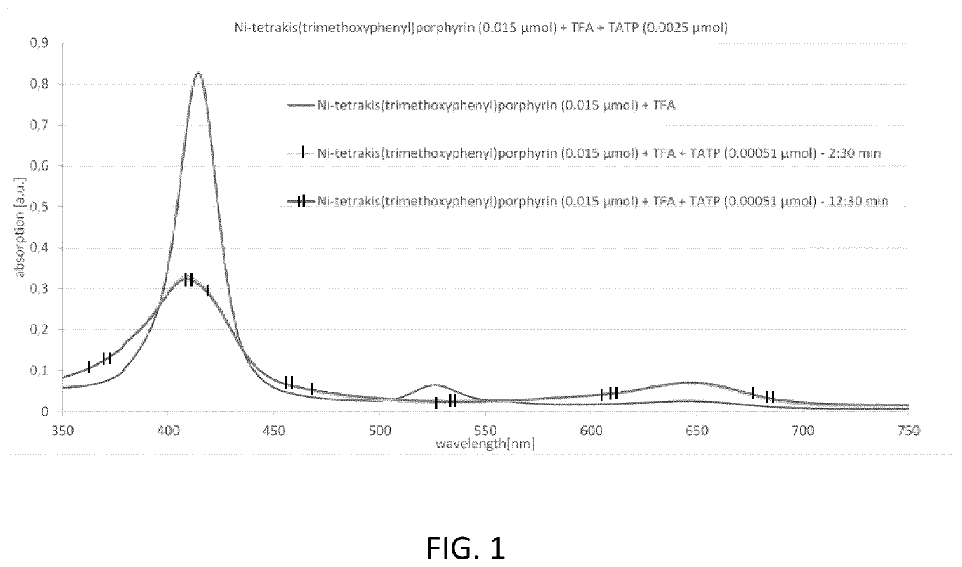

FIG. 2 which shows test stripes as an embodiment of the invention.

### DETAILED DESCRIPTION OF THE PREFERRED EMBODIMENTS

The invention provides an analytical process for detecting peroxide-oxyhalide anion- and nitrogen-based explosives, with high sensitivity and without pre-treatment of a sample prior to a color reaction that can be detected visually, preferably without spectrophotometric detection. The analytical process preferably can be performed at ambient conditions within a short time, e.g. within 30 s, within 20 s, or less. Preferred embodiments provide a simple-to-use device containing the reactants for use in the analytical process.

The present invention relates to an analytical process for detecting several classes of compounds, especially peroxide-, oxyhalide anion-, and nitrate-based compounds, which are explosives. These compounds especially include: 1. Peroxide-based compounds, especially peroxide-based explosives, e.g. triacetone triperoxide (TATP), diacetone diperoxide (DADP), methyl ethyl ketone peroxide (MEKP), hexamethylene triperoxide diamine (HMTD), 2. Oxyhalide anions, especially chlorates (e.g. sodium chlorate (NaClO₃), potassium chlorate (KClO₃) or bromates, e.g. sodium bromate (NaBrO₃), 3. Inorganic nitrates (nitrate salts) e.g. potassium nitrate (KNO₃), ammonium nitrate (NH₄NO₃), urea nitrate (NH₂COHNH₂⁺·NO₃⁻). The analytical process of the invention has the advantage of having a high sensitivity, it does not require sample preparation, and it detects the most relevant classes of explosive compounds with the same reagent in one step by a simple chemical color test.

Preferred embodiments provide an analytical process, which preferably is a one-step process, for detecting in a sample as the analytes peroxide-based explosives, oxyhalide anion-based explosives, and/or nitrate-based explosives, the process comprising contacting a sample suspected of containing a peroxide-based compound, especially a peroxide-based explosive, an oxyhalide anion-based compound, especially an oxyhalide-based explosive, a nitrate salt, especially a nitrate-based explosive, with a composition comprising or consisting of a Ni-porphyrin, an acid and preferably an acid-stable solvent. The process does not comprise a separate step of hydrolysing the sample suspected of containing a peroxide-based explosive prior to contacting the sample with the composition containing the Ni-porphyrin. Further, the invention provides the composition comprising the Ni-porphyrin, an acid, optionally an acid-stable solvent, for use as an analytical device in the analytical process. The acid can be present in a mixture with the Ni-porphyrin.

The analytical process and the composition have the advantage of allowing a differentiation between the explosives, as peroxide-based explosives and oxyhalide anions are indicated by the Ni-porphyrin finally generating green color, and that nitrate-based and oxyhalide anion-based explosives are indicated by the Ni-porphyrin initially generating green color and finally generating brown color.

Further, the invention provides a device for use in the analytical process, the device comprising the composition comprising or consisting of a Ni-porphyrin, an acid and preferably an acid-stable solvent and preferably a carrier, e.g. a porous carrier, which carrier holds the Ni-porphyrin, acid and acid-stable solvent, wherein the carrier e.g. is a porous and/or swelling liquid adsorbing material that forms a gel, e.g. a membrane of an acid-stable material, e.g. of a synthetic polymer or cellulose, so that the carrier forms a supporting material for a liquid composition of the invention.

The process and the composition of the invention have the advantage of detecting explosives in one process step, because the Ni-porphyrin is acid-stable and the acid decomposes the peroxide-based explosive to generate a hydroperoxide, or hydrogen peroxide, which react with the Ni-porphyrin to change its color.

It was found that the process is extremely sensitive towards peroxides, because. one TATP molecule will result in the conversion of 6 Ni-porphyrin molecules to the corresponding radical cation, which gives a color change from red to green. Nitrate- and oxyhalide anion based compounds accept two electrons from the porphyrin forming the brown-colored dication of the porphyrin. A high sensitivity is also provided by the fact that Ni-porphyrins exhibit extremely high molar extinction coefficients of their Soret bands. Therefore, only small amounts of the porphyrin need to be used in the invention for visual detection of a color change.

In contrast to the prior art using separate activation of the explosives, e.g. by separate acid or base hydrolysis or UV irradiation, with subsequent detection by a color reaction, the analytical process of the invention detects the above listed explosives in a reaction that only requires contacting the sample suspected of containing the peroxide-oxyhalide- or nitrogen based explosive with the composition comprising or consisting of Ni-porphyrin, acid and optionally acid-stable solvent.

The acid is a free acid that is present in admixture with the Ni-porphyrin and with the optional solvent, but which free acid is not covalently linked to the Ni-porphyrin. The free acid, e.g. in the form of a liquid acid, is one acid or a mixture of at least two acids.

The Ni-porphyrin, preferably in at least one meso position, has electron-donating substituents, which are aryl groups, e.g. aromatic groups containing at least one phenyl ring, which optionally are substituted with at least one methoxy group, or substituted amino group, or alkyl groups, each having e.g. 1, 2, 3, 4, 5, 6, 7, 8, 9, 10, 11, or 12 carbon atoms, linear or branched or cyclic. The Ni-porphyrin can have its meso positions unsubstituted, or the Ni-porphyrin can have one, two, three or all four of its meso positions substituted, preferably with an electron-donating group, which can be the same or independently a different one for each meso position.

Generally, the acid in all embodiments preferably has a pKₐ value of at maximum 1.2, e.g. a pKₐ value of below 1.2, e.g. of −2.8 to 0.23. For example, p-toluenesulfonic acid has a pKₐ value of −2.8, trichloroacetic acid has a pKₐ value of 0.7. Generally preferred, the acid has a pKₐ in the range from −1.2 to 0.23. The pKₐ value preferably is determined at 20° C. in water. A pKₐ value of at maximum 1 is preferred, because it results in decomposition of peroxide-based explosives that have a cyclic structure such as TATP within 30 s or shorter, e.g. at 20° C. The pKₐ of the acid should preferably not be below −4 (e.g. triflic acid pKₐ −5.1) to prevent decomposition of the Ni-porphyrin.

In an embodiment, the acid is a compound, which is in admixture with the Ni-porphyrin, preferably in an acid stable solvent. The acid can be a strong acid, e.g. having a pKₐ of −1.2 to 1.2 or to 0.23. The acid preferably is selected from trifluoroacetic acid (pKₐ 0.23), pentafluoro propionic acid (pKₐ 0.5-1.2), heptafluoro butyric acid (pKₐ 1.2) or perfluoropentanoic acid (pKₐ 0.5-1.2) preferably perfluoropentanoic acid, and mixtures of at least two of these.

The acid stable solvent preferably is a solvent that stabilizes cations and radical cations. The acid stable solvent can be an organic halogenated solvent, e.g. methylene chloride, chloroform, 1,1-dichloroethane, 1,2,3-trichloropropane, 1,2,3-trichloropropane, 1,1,2,3-tetrachloropropane, 1,1,1,2,3-pentachloropropane, 1,1,2,3,3-pentachloropane, fluorinated C₂- to C₆-alcohols, preferably hexafluoro isopropanol (HFIP), or sulfolane or mixtures of at least two of these.

In chlorinated solvents, the acid preferably is trifluoroacetic acid (TFA), pentafluoro propionic acid, heptafluorobutyric acid, or perfluoropentanoic acid. In chlorinated solvents, in fluorinated alcohols, e.g. in HFIP, or in sulfolane the acid can be toluenesulfonic acid. Preferentially, higher boiling solvents are combined with higher boiling acids to prevent rapid evaporation in some embodiments.

Generally, the composition comprising the Ni-porphyrin and acid and optionally a solvent can be held on, e.g. adsorbed on or contained in a solid carrier, which can for example be a porous substance or a gel. The porous substance can e.g. be silica or zeolite or cellulose. The gel can e.g. be an acid stable polymer such as polyvinyl alcohol (PVA) or polyvinyl pyrrolidone (PVP). Preferentially, a gel is used, which contains the acid in the polymer backbone. These gels are commercially available as strongly acidic cation exchange resins such as Amberlite® R-120 or Dowex® 50 WX-4.

As an alternative to holding the composition on a carrier, the composition can be contacted in the form of droplets with the sample, e.g. by spraying a liquid composition containing the Ni-porphyrin, preferably contacting the composition on a solid carrier, preferably a white carrier, which can e.g. be a wall of a container suspected of containing a sample containing an explosive, or a paper, a plastic, or a glass surface arranged inside the sample or in the vicinity of the sample. In this embodiment, it is preferred that the acid is a substituent to the Ni-porphyrin, which has the advantage of being less irritating, of the Ni-porphyrin that is covalently linked to the acid being less volatile, and/or of being less corrosive than a free acid.

As a carrier containing free acid in the form of a matrix-bound acid, test stripes can be produced by using a strongly acidic cation exchange resin impregnated with the Ni-porphyrin dissolved in halogenated solvent. Dipping the test stripes into the headspace of TATP or EGDN, or direct contact with traces of peroxide- or nitrogen-based explosives, such as triacetone triperoxide (TATP), diacetone diperoxide (DADP), hexamethylene triperoxide diamine (HMDT), methyl ethyl ketone peroxide (MEKP), potassium chlorate (KClO₃, potassium bromate (KBrO₃), potassium nitrate (KNO₃), ammonium nitrate (NH₄NO₃), urea nitrate (NH₂COHNH₂⁺·NO₃⁻), or nitrourea (NH₂CONHNO₂) leads to a color change from red to green. In case of the nitrogen-based explosives a subsequent color change from green to brown is observed. Detection of explosives is also possible in mixtures such as black powder (KNO₃/sulphur/charcoal) or pyrotechnic compositions (e.g. KClO₃/Al-powder).

In another aspect of the invention, acid stable porous polymer membranes such as polypropylene membranes, PVC membrane filters, or porous polyvinylidene fluoride, or cellulose, impregnated with the Ni-porphyrin dissolved in pure acid such as trifluoroacetic acid or preferentially higher boiling acids e.g. pentafluoro propionic acid are used as test stripes.

Generally, test stripes can comprise an inert base material, e.g. a plastic stripe, with an attached porous substance or gel for accommodating the Ni-porphyrin in mixture with a free acid. The test stripes can be in combination with free acid and/or an acid stable solvent contained in a container suitable for dispensing liquid, preferable is a separate container suitable for dispensing liquid, as a kit-of-parts. In the alternative to the Ni-porphyrin being comprised in a test stripe, the Ni-porphyrin in mixture with a free acid can be present in a liquid composition, preferably in an acid-stable solvent, as a kit-of-parts. A kit-of-parts contains the compounds and is suitable for carrying out the analytical process of the invention.

Dipping the test stripes into the headspace of TATP or direct contact with traces of peroxides, oxyhalide salts, or nitrate salts leads to a color change from red to green.

In another aspect of the invention, the acid-substituted Ni-porphyrin dissolved in a solvent is sprayed onto a surface suspected to be contaminated with an explosive. A change of color from red to green would indicate the presence of an explosive. Alternatively, one can take a swab of a surface using a wiping cloth and apply the spray to the wiping cloth.

The Ni-porphyrin can comprise or consist of Structure 1, which shows an embodiment in which the Ni-porphyrin is substituted with aryl substituent groups (Ar) in all four of the meso positions. Exemplary aryl substituent groups can be independently selected from a phenyl or other aromatic rings (Ar), which aryl group optionally is further substituted by at least one electron donating group, herein shown as a methoxy group, e.g. as shown one methoxy group or three methoxy groups, and shown as three methyl groups. Exemplary aryl groups (Ar) are the phenyl group, a methoxy phenyl group, a tri-methoxy phenyl group, and a trimethyl phenyl group. The aryl group can be bound by any of its carbon atoms to the carbon in meso position of the Ni-porphyrin.

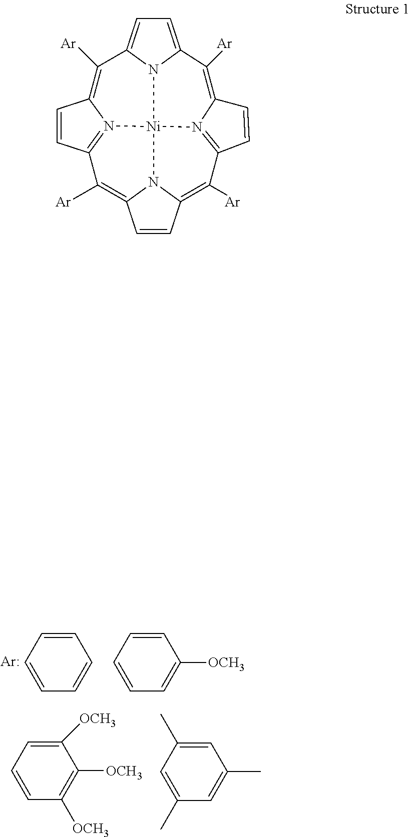

In an embodiment, although less preferred, the aryl substituent groups do not carry an electron donating group. In this embodiment of the aryl substituent groups being free from electron donating groups, the analytical process is slower to generate the indicating color, and the process is less sensitive.

Structure 1 is suitable for an embodiment, in which the composition comprises the Ni-porphyrin and, as a compound in admixture with the Ni-porphyrin, an acid, optionally a solvent.

Optionally, the process can consist of contacting the sample with the composition, or the process can comprise an additional step of destroying free hydrogen peroxide from the sample prior to contacting the sample with the composition in order to reduce or eliminate the influence of free hydrogen peroxide. For hydrolysing free hydrogen peroxide, prior to contacting the sample with the composition comprising Ni-porphyrin, the sample can be contacted with a reactant having activity to disintegrate free hydrogen peroxide. Accordingly, the device can optionally comprise a flow path in which the composition comprising the Ni-porphyrin is arranged to receive a sample, wherein upstream of this composition there is arranged a reactant having activity to disintegrate free hydrogen peroxide. The reactant can e.g. be an inorganic catalyst, e.g. KMnO₄ or MnO₂. The reactant can be immobilized on a porous carrier that optionally spans the cross-section of the flow path.

A flow path can e.g. be provided by a wicking action material, e.g. a porous material, and/or by a duct through which a sample can migrate, e.g. by capillary action or by positive or negative pressure applied to the duct.

The invention provides an analytical process for detecting a compound which is an explosive, comprising contacting a sample suspected of containing a peroxide-based compound, a nitrate-based compound, or an oxoanion based compound with a composition comprising or consisting of a Ni-porphyrin, and a free acid.

In the analytical process the peroxide-based explosive can be at least one of triacetone triperoxide (TATP), diacetone diperoxide (DADP), methyl ethyl ketone peroxide (MEKP) and/or hexamethylene triperoxide diamine (HMTD), wherein the nitrate-based explosive is potassium nitrate (KNO₃), sodium nitrate (NaNO₃), ammonium nitrate (NH₄NO₃), and/or urea nitrate (NH₂COHNH₂⁺·NO₃⁻).

Optionally, the composition contains an acid-stable solvent.

Optionally, the sample is a gaseous sample taken from a container or from an environment suspected of containing a peroxide-based explosive.

Optionally, the composition comprises or consists of a Ni-porphyrin, an acid and optionally an acid-stable solvent, is held by a carrier, which is a porous acid-stable material and/or swelling liquid adsorbing acid-stable material that forms a gel, the carrier forming a supporting material for a liquid composition containing the Ni-porphyrin and the acid.

Optionally, the composition comprising or consisting of a Ni-porphyrin, an acid and optionally an acid-stable solvent is liquid and is contacted with the sample in the form of droplets.

Optionally, a portion of the sample is contacted with the composition comprising the Ni-porphyrin and a strong acid in order to detect all peroxides, and a portion of the sample is contacted with the composition comprising the Ni-porphyrin and a weaker acid in order to detect only hydrogen peroxide.

Peroxide-based compounds are indicated by the Ni-porphyrin finally generating green color, and nitrate-based compounds are indicated by the Ni-porphyrin generating green color and finally generating brown color.

The composition for use in an analytical process is for use in detecting a peroxide-based compound, a nitrate-based compound, wherein the composition comprises or consists of Ni-porphyrin, a free acid having a pKₐ of −1.2 to 0.23, and optionally an acid-stable solvent.

Optionally, the composition is held on a carrier that is porous and/or swelling.

Optionally, the composition is a solution in an acid-stable solvent.

Optionally, in the composition the Ni-porphyrin is present on a porous carrier that is attached to a plastic strip, in combination with a container containing the acid as a liquid, optionally the acid being in mixture with an acid-stable organic solvent, optionally in combination with a container containing a liquid superbase, in a kit-of-parts.

Optionally, in the composition the Ni-porphyrin is in a mixture with a free acid having a pKa of −1.2 to 0.23, which Ni-porphyrin and free acid are present in an acid-stable organic solvent in a container, in a kit-of-parts.

Preferred embodiments of the invention will now be discussed with respect to experiments and drawings. Broader aspects of the invention will be understood by artisans in view of the general knowledge in the art and the description of the experiments that follows.

### Example 1: Detecting Solid TATP from the Head Space

A plastic stripe (90×5 mm) equipped with a cellulose pad (5×5 mm) at one end, impregnated with Ni-tetrakis(3,4,5-trimethoxyphenyl)porphyrin by dropping 5 μL of a 1 mM solution of the porphyrin in toluene onto the cellulose pad and then the solvent is removed by evaporation. The stripe prepared in this way is moistened with a drop of 5-10 μL of perfluoropentanoic acid. The addition of the acid to the Ni-porphyrin compound of the invention herein is also referred to as activating the Ni-porphyrin or the stick. The activated stick is brought into close proximity, ca. ˜3 mm to crystals of TATP present on an uncovered surface or into the headspace of TATP present in a small glass vial. After 15 s the red color of the pad turns into green. Assuming a saturation vapour pressure in the head space of the solid explosive and a volume of 3 mL of gas, 8 nmol of TATP can be detected.

### Example 2: Detecting Nitrate Salts (e.g. NH₄NO₃, KNO₃, NaNO₃) from the Head Space

Traces of nitrate salts (e.g. NH₄NO₃, KNO₃, NaNO₃) each separately present on an uncovered surface, or in a small glass vial are treated with a drop (5-10 μl) of perfluoropentanoic acid. After several seconds, the activated plastic stripe prepared as in Example 1 is brought into close proximity (˜3 mm) to one of the compounds present on the surface or into the head space of the glass vial. The color of the pad turns from red to brown. The detection of nitrate salts by the invention is obtained also when the nitrate salts are present in solution or in a dry mixture, e.g. with sulfur and charcoal as in black powder.

### Example 3: Detecting Peroxides (e.g. HMTD, TATP), Chlorates (e.g. KClO₃, NaClO₃), and Bromates (e.g. KBrO₃, NaBrO₃) in the Solid State

The activated stripe (prepared as in Example 1) is separately brought into contact with traces of solid HMDT, TATP, or chlorate by swiping a contaminated surface. The color of the stripe immediately changes from red to green at the spots, where the pad comes into contact with traces of the solid explosive. These green spots spread out and within about 30 seconds the pad turns green. 10 μl of a 12.65 UM solution of TATP dissolved in dichloromethane were dropped as a round spot onto the previously prepared test stripe. After one minute the test stripe turned light green. Using this method, it is possible to detect 0.125 nmol (28 ng) TATP.

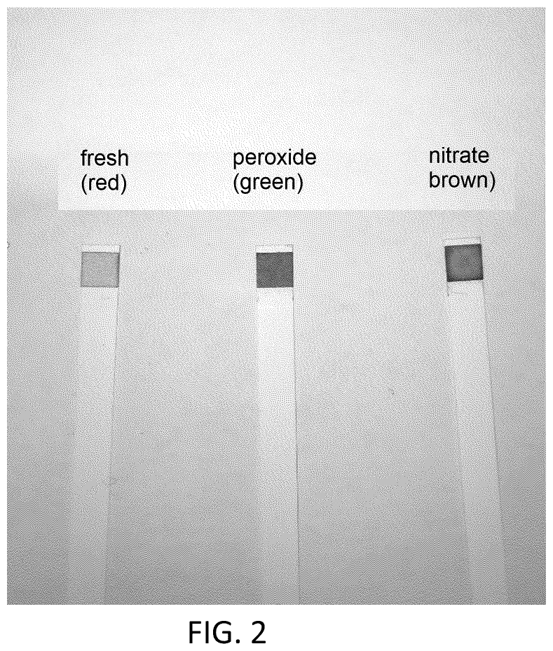

FIG. 2 shows a plastic stripe with a terminally attached cellulose pad impregnated with Ni-porphyrin according to the invention, prior to contact with a compound to be detected (fresh (red)), and a plastic stripe containing the same Ni-porphyrin, activated by adding acid and after contact with peroxide (peroxide (green)), and a plastic stripe containing the same Ni-porphyrin activated by adding acid and after contact with nitrate (nitrate (brown)).

### Example 4: Detecting Nitrate Salts (e.g. NH₄NO₃, KNO₃, NaNO₃) in the Crystalline State

The activated stripe prepared as in Example 1 is brought into contact with separate traces of solid nitrate salts by swiping a contaminated surface. The color of the stick immediately changes from red to green at the spots, where the pad comes into contact with traces of the explosive. These green spots turn brown and spread out over the pad.

### Example 5: Detecting HMTD and TATP from Solution

For detection of HMTD and/or TATP in solution, 300 μL of Ni-tetrakis(3,4,5-trimethoxyphenyl)porphyrin (50 μM in CH₂Cl₂) and 50 μL trifluoroacetic acid (13 M) were transferred into a sample tube. After adding 10 μL of a 25.3 μM solution (HMTD or TATP separately in CH₂Cl₂), the solution turned green within several minutes due to the formation of the porphyrin π-radical cation. It was calculated that the process can detect 0.18 nmol (40 ng) TATP or (50 ng) HMTD as a color change from red to green. Analogously, the detection limit of potassium perchlorate (KClO₃) was determined as 270 ng, and the detection limit of ammonium nitrate (NH₄NO₃) is 85 ng.

### Example 6: Practical Implementation of the Test

A plastic stripe (90×5 mm) is equipped with a cellulose pad (5×5 mm) at one end. The pad is moistened with a solution of the Ni-porphyrin in an organic solvent. The solvent is evaporated leaving the dry pad uniformly impregnated with the red colored porphyrin. This stick is storable for extended periods of time (>2 years at room temperature in a dry environment). Prior to application, the stripe is activated with a drop (5-10 μL) of perfluoropentanoic acid applied onto the pad. The stripe now remains active and ready to use for about 15 min. The acid can be applied from a dropper bottle, or released from a reservoir above the pad by mechanical force. In these examples, the following detection limits could be determined: for TATP: ˜40 ng, for KClO₃: ˜270 ng, for NH₄NO₃: ˜85 ng, for urea nitrate: ˜350 ng, for HMTD: ˜50 ng.

### Comparative Example 1

For comparative purposes, electron rich Ni-porphyrins (Ni-tetra(4-hydroxyphenyl)porphyrins) have been prepared according to M. K. Chahal, M. Sankar, Dalton Trans. 2016, 45, 16404-16412 and used as sensors for cyanide (CN⁻), fluoride (F⁻) and picric acid according to M. K. Chahal, M. Sankar, Dalton Trans.2017, 46, 11669-11678. To the Ni-porphyrins used in these publications, trifluoroacidic acid (TFA) and TATP and HMDT was added. After one minute at room temperature an unspecific reaction took place. Hence, these porphyrins described by Chahal et al. are not suitable for the detection of peroxide-based explosives.

### Comparative Example 2

Ni-tetra(carboxyphenyl)porphyrins with CO₂H groups directly attached to the meso phenyl rings in 2, 3 or 4 position (B. Du, A. Langlois, D. Fortin, C. Stern, P. D. Harvey, J. Clust. Sci. 2012, 23, 737-751) are not suitable, and it is assumed that this is because the carboxylic group is not sufficiently acidic (pKₐ 4.2) to cleave peroxide or nitrate based explosives. Ni-tetrakis(4-carboxyphenyl)porphyrin (CAS #136300-60-2) was synthesized (B. J. Johnson et al. Sensors 2010, 10, 2315-2331) and dissolved in methylene chloride. TATP and HMDT was added. No color change was observed.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. An analytical process for detecting explosive compounds by a color reaction, comprising contacting a sample suspected of containing a peroxide-based compound, a nitrate-based compound, an oxoanion-based compound, an oxyhalide anion-based compound, a chlorate or a bromate with a composition comprising a Ni-porphyrin and a strong free acid having a pKₐ of −2.8 to 1.2 and checking for the color reaction.

2. The analytical process according to claim 1, wherein the peroxide-based explosive is at least one of triacetone triperoxide (TATP), diacetone diperoxide (DADP), methyl ethyl ketone peroxide (MEKP) and/or hexamethylene triperoxide diamine (HMTD), wherein the nitrate-based explosive is potassium nitrate (KNO₃), sodium nitrate (NaNO₃), ammonium nitrate (NH₄NO₃), urea nitrate (NH₂COHNH₂⁺·NO₃⁻), and the chlorate is sodium chlorate (NaClO₃) or potassium chlorate (KClO₃).

3. The analytical process according to claim 1, wherein the composition contains an acid-stable solvent.

4. The analytical process according claim 1, wherein the sample is a gaseous sample taken from a container or from an environment suspected of containing a peroxide-based explosive.

5. The analytical process according to claim 1, wherein the composition comprising or consisting of a Ni-porphyrin, the acid and optionally an acid-stable solvent, is held by a carrier, which is a porous acid-stable material and/or swelling liquid adsorbing acid-stable material that forms a gel, the carrier forming a supporting material for a liquid composition containing the Ni-porphyrin and the acid.

6. The analytical process according to claim 1, wherein the composition comprising or consisting of a Ni-porphyrin, an acid and optionally an acid-stable solvent is liquid and is contacted with the sample in the form of droplets.

7. The analytical process according to claim 1, wherein a portion of the sample is contacted with the composition comprising the Ni-porphyrin and the strong acid in order to detect all peroxides, and a portion of the sample is contacted with the composition comprising the Ni-porphyrin and a weaker acid in order to detect only hydrogen peroxide.

8. The analytical process according to claim 1, wherein peroxide-based compounds are indicated by the Ni-porphyrin finally generating green color, and that nitrate-based compounds are indicated by the Ni-porphyrin generating green color and finally generating brown color.

9. The analytical process according to claim 1, wherein the acid is selected from trifluoroacetic acid (pKₐ 0.23), pentafluoro propionic acid (pKₐ 0.5-1.2), heptafluoro butyric acid (pKₐ 1.2), perfluoropentanoic acid, perfluorohexanoic acid (pKₐ 0.5-1.2), and mixtures of at least two of these.

10. Analytical process according to one claim 1, wherein the composition consists of the Ni-porphyrin and the strong free acid having a pKₐ of −2.8 to 1.2.

11. A kit for carrying out the analytical process of claim 1, comprising a Ni-porphyrin, a free acid having a pKₐ of −2.8 to 1.2, and optionally an acid-stable solvent, wherein the Ni-porphyrin is present on a porous and/or swelling carrier, optionally attached to an inert base material, in combination with a container containing the acid as a liquid, optionally the acid being in mixture with an acid-stable organic solvent, or
the Ni-porphyrin is in a mixture with the free acid, which Ni-porphyrin and free acid are present in an acid-stable organic solvent in a container.

12. The kit according to claim 11, wherein the kit-of-parts consists of the Ni-porphyrin and the free acid, optionally attached to an inert base material.

13. The kit according to claim 11, wherein the free acid is selected from pentafluoro propionic acid, heptafluoro butyric acid, perfluoropentanoic acid, perfluorohexanoic acid, and mixtures of at least two of these.
